(19) United States
(12) Patent Application Publication
(10) Pub. No.: US 2020/0352440 A1
(43) Pub. Date: Nov. 12, 2020

(54) MEDICAL SYSTEM FOR REMOTE MONITORING, ANALYSIS AND PROGNOSIS OF THE PATIENT'S CONDITION BY FIRST-LEAD ELECTROCARDIOGRAM SERIES AND COMPUTER-ASSISTED METHOD FOR MONITORING, ANALYSIS AND PROGNOSIS OF THE PATIENT'S CONDITION

(71) Applicant: ZAKRYTOE AKTSIONERNOE OBSCHESTVO "EC-LEASING", Moscow (RU)

(72) Inventors: Alexander Viktorovich SHMID, Moscow (RU); Andrey Alexandrovich BEREZIN, Moscow (RU); Maxim Alexandrovich NOVOPASHIN, Ivanovskaya obl. (RU)

(73) Assignee: ZAKRYTOE AKTSIONERNOE OBSCHESTVO "EC-LEASING", Moscow (RU)

(21) Appl. No.: 16/760,656

(22) PCT Filed: Jan. 31, 2018
(86) PCT No.: PCT/RU2018/000054

§ 371 Date: Apr. 30, 2020

(51) Int. Cl. A61B 5/00; A61B 5/04; G16H 10/60; G16H 40/67; G06K 9/62

(52) U.S. Cl. CPC A61B 5/0006; A61B 5/04012; G06K 9/6218; G16H 10/60; G16H 40/67; A61B 5/7275

(57) ABSTRACT

Medical system for remote monitoring, analysis and prognosis of patient's condition by series of at least first-lead electrocardiograms (ECGs), comprises: unit for gathering for each patient a series of ECGs; unit for extracting from the gathered database an ECG subset of a target group of patients, in accordance with at least one of identifiers according to examination purpose; unit for clustering the extracted ECG subset based on the feature of similarity of their forms; unit for determining an ECG form similarity measure in the space of ECG form features associates each ECG with a discretized ECG (DECG), which is a point having coordinates in at least one space of ECG form features; unit for generating STANDARDS for each cluster; unit for assessing variation in patient's condition over time by temporal variation in distances between each newly received DECGs in patient's DECG series and assigned at least one STANDARD.

### TECHNICAL FIELD

The present invention relates to medicine and, more specifically, to a medical system for remote monitoring, analysis and prognosis of the patient's condition by series of at least first-lead electrocardiograms (ECG) recorded for each of the observed patients, and to a computer-assisted method for monitoring/analysis and prognosis of the patient's condition by first-lead electrocardiogram series (ECGs).

The invention can be used for improving the accuracy of assessment of the patient's condition by automatically providing more information (Big Data) to ECG specialists and ECG transcriptionists and for evaluating the effectiveness of medication treatment based on a cardiogram and/or pulse wave.

### BACKGROUND OF THE INVENTION

Cardiovascular diseases remain the leading cause of death worldwide, and coronary heart disease (CHD) contributes most to the overall picture of mortality. In Europe, mortality from cardiovascular diseases in 2013 was about 49%. In Russia in 2009, the CHD accounted for 28.9% of all deaths.

This fact causes the urgency of the problem of predicting the condition in patients with chronic coronary heart disease (CCHD) in order to choose the proper therapeutic approach. The amount of therapy required to a particular CHD patient should be primarily determined from individual risk which can vary significantly.

The high-priority general scientific application is the prognosis, which, using mathematical models and methods, would enable determining the tolerance level of the human body to effects of various factors in order to specify the predisposition to a particular disease, and if the predisposition has already arisen, predicting the specifics of its future progression and the outcome.

To successfully solve this problem, it is necessary to provide an algorithm available to any practitioner, which would enable dividing patients into risk groups and selecting the optimal treatment approach for a specific patient from the prognosis viewpoint. To date, there is no uniform method for calculating the risk of death and cardiovascular events.

A method for predicting the progression of coronary heart disease is disclosed in patent RU 2391044 (published Oct. 6, 2010). The method involves clinical and electrocardiographic examinations, Holter monitoring, echocardiography, bicycle ergometry, heart rate variability analysis, signal-averaged electrocardiography, determination of QT interval variance. The method uses a staged examination of patients, where the first stage comprises clinical examination, the second stage—Holter ECG monitoring, the third stage—echocardiography, the fourth stage—stress test, and the fifth stage comprises analysis of the average ECG heart rate variability signal. At each stage, methods of multivariate statistical analysis distinguish high-risk predictors of occurrence of the events that are life-threatening for patients with chronic coronary insufficiency. Unfavorable prognosis for coronary heart disease is predicted if the first stage identifies: presence of angina pains, irregular heartbeat, syncope condition; arterial hypertension, diabetes mellitus, smoking, physical inactivity, hyperlipidemia, psycho-emotional stress, presence of criteria for cicatricial changes and evidences of ischemia indicants, presence of ventricular rhythm disturbances, including high gradations, lengthening of QT interval in analysis of QT dispersion at resting electrocardiography. Unfavorable identifiers at the second stage include: daily duration of all ischemic episodes more than 60 minutes; presence of painless episodes of ischemia; maximum depth of ST segment depression of more than 3 mm; the number of heart contractions at the beginning of pain episodes less than 110 per minute; the number of heart contractions at the beginning of painless episodes less than 100 per minute; presence of extrasystole of high IV, V gradations, runs of ventricular tachycardia; presence of ventricular extrasystole. Unfavorable identifiers at the third stage include: finite diastolic size of more than 6.0 cm, ejection fraction of less than 40%, presence of hypo- and akinesia zones, evidences of aneurysm. Unfavorable identifiers at the fourth stage include: decrease in airway tolerance to physical activity of less than 210 conventional units, maximum heart rate at the beginning of ischemic episodes less than 100 per minute; presence of ventricular arrhythmias associated with transient episodes of ischemia, the number of leads with ST segment depression more than 6. Unfavorable identifiers at the fifth stage include: presence of late ventricular potentials, SDNN values less than 21 ms, BB50 less than 10 beats/min, RMSDD less than 15 ms, LF/HF more than 1.7 conventional units, triangular index less than 15.

Based on the results of phased examination, the probability of a favorable or unfavorable CHD progression is calculated using the length of vectors of CHD course variants and, accordingly, the probability of a high or low risk of death of patients is predicted.

This method is unable to provide effective diagnosis of patients outside the healthcare center when determining the patient's condition and rendering aid to both the patient in express assessment of his current condition and the clinician in monitoring the patient's treatment outside the healthcare center.

RU 2615721 B2 discloses a device for cardiographic monitoring of the patient's condition, comprising a monitor, an interface, at least one electric cardio signal sensor (ECG electrode) mounted on a patient and designed to pick up electric cardio signals from the patient's body, connected by an output to input of a primary signal processing unit, the other input of this unit being connected to output of the time sampling unit, and output of the primary signal processing unit is connected to a channel switching unit. Outputs of the channel switching unit are connected to a discrete Fourier transform unit which outputs values of the amplitude, frequency and phase of harmonics of the signal under study, and to a patient data input unit. Harmonics are processed in a cardiogram register that stores and outputs harmonic amplitudes of the signal under study for required amount of time. The harmonic amplitudes are input into a cardiogram image identifier, which compares the image received from the ECG electrode, taking into account the confidence intervals and a certain degree of reliability, with images from the cardiogram image base. Output of the identifier is connected to input of a unit for recording the states and analyzing their dynamics, where a patient's disease diagnosis is formed on the basis of data of the cardiogram images from all ECG electrodes by comparing the set of cardiogram images from ECG electrodes with a disease diagnosis characterizing set from the cardiological diagnosis base, taking into account confidence intervals and a certain degree of reliability. The same unit determines the diagnosis reliability level, the diagnosis dynamics depending on the previous examination of the patient, and the diagnosis determination time. The data is displayed on the monitor, transferred to the interface for storage and study at other technical means and to the patient data input unit, where they are stored in the respective patient's archives.

The device is unable to provide effective diagnosis of patients outside the healthcare center when determining the patient's condition and assisting both the patient in express assessment of his current condition and the clinician in monitoring the patient's treatment outside the healthcare center.

Most closely related to the present invention is CardioQVARK portable system (O. Yu. Pidanov, “The first experience of personal ECG monitoring in patients after thoracoscopic ablation of the left atrium,” Vestnik Sovremennoy klinicheskoy meditsiny, 2017, v.10, issue 6, p.p. 24-30). The system serves as a tool for cardiovascular system monitoring in patients after thoracoscopic ablation due to atrial fibrillation. 5 minute ECG recordings were taken with CardioQVARK portable monitor in hospital patients from 1st to 5th day after the surgery, and data was transmitted by mobile Internet via cloud service to CardioQvark Doctor application. The hospital stage has demonstrated convenience and ease of use of the personal CardioQVARK monitor when monitoring patients in the period after surgical treatment for atrial fibrillation. Remote monitoring by CardioQVARK allows continuous observation of patients.

Use of iPad CardioQVARK Doctor application enables gathering of a research base, which is convenient for quick access, and quick analysis of the cardiogram QT, PQ interval for finding antiarrhythmic medication dose.

Test investigation comprised monitoring ECG patients daily for 2 months when they were out of the hospital. 64 ECG recordings with a total duration of 262 minutes were obtained. Of the obtained records, 61 (95.3%) were suitable for analysis. The investigation provided for recording ECG daily in the morning, and recording and transmitting data when a sensation of irregular heartbeat occurred i.e. event monitoring.

The monograph states that the use of CardioQVARK device precludes the complete omission of using bedside monitors, ECG devices, Holter monitors, etc.

The advantage of long-term and, even better, permanent electrocardiographic (ECG) monitoring cannot be doubted. However, in real clinical practice, the use of monitors is extremely limited due to their high cost.

When an arrhythmia attack occurs, its recording may be impossible simply because the attack can stop on its own before the patient reaches the ECG office or the ambulance arrives. Under such conditions, it becomes extremely attractive to monitor cardiac rhythm disturbances with the ability to transmit the results to a specialist at a distance using current communication technologies.

The above system is unable to provide prognosis of the observed patient's condition.

### SUMMARY OF THE INVENTION

The object of the present invention is to provide a medical system for remote analysis of cardiac data obtained from at least one patient outside the healthcare center, analysis and prognosis of the patient's condition based on series of at least first-lead electrocardiograms (ECG) recorded for each of the observed patients, which can quickly and/or reliably overcome the aforementioned problems of the prior art.

Another object of the present invention is to provide a computer-assisted method for monitoring, analysis and prognosis of the patient's condition based on series of at least first-lead cardiac electrocardiograms (ECGs) and other cardiological signals synchronized with ECG, using this medical system, which can enable efficient analysis, diagnosis and prognosis of the patient's condition and render aid to the clinician in treatment planning.

The advantage provided by the present invention comprises the provision of effective diagnosis of patients outside the healthcare center when determining the patient's condition and assistance to both the patient in express assessment of his current condition and the clinician in monitoring the progression of patient's treatment outside the healthcare center, including the assessment of cardiotoxicity in the use of medications, not only cardiac ones.

Therefore, according to a first aspect of the present invention the object is attained by providing a medical system for remote monitoring, analysis and prognosis of the patient's condition by series of at least first-lead electrocardiograms (ECGs) recorded for each patient from a plurality of observed patients, comprising:

a unit for gathering in a database of said medical system for each of the observed patients a series of his electrocardiograms (ECGs) marked with patient identifiers, generated by adding each newly recorded patient's ECG marked with said identifiers to the patient's ECG series gathered previously throughout the entire observation time;

a unit for extracting on doctor's request from the gathered database an ECG subset of a target group of patients, which are combined in said system into the target group in accordance with restrictions specified in the doctor's request and at least one of said identifiers in accordance with the examination purpose;

a unit for clustering the extracted ECG subset, which sequentially combines, for at least one target group of patients combined by at least one feature, all the ECGs extracted to the subset to at least one cluster based on the feature of similarity of their forms using a clustering method;

a unit for determining an ECG form similarity measure in the space of ECG form features, which associates each ECG from the database of the medical system with a discretized ECG (DECG), which is a point having coordinates in at least one space of ECG form features, chosen by the system operator, wherein coordinates of the point in said space of ECG form features carry information about the original ECG form, and the ECG form similarity measure between any ECG pairs is specified as the distance between their corresponding pairs of DECG points in this space;

a unit for generating STANDARDS, which generates, for each cluster formed by the unit (3), a corresponding STANDARD by forming a set of DECG points corresponding to the cluster ECGs, and calculates characteristics of the formed DECG set, including: coordinates of the center of gravity of the formed DECG set, radius of the sphere or center of the polygon at the center of gravity of the DECG set, including all the DECG sets;

a unit for assigning, on doctor's request, at least one STANDARD in accordance with the examination purpose from a plurality of generated STANDARDS for subsequent assessment of the patient's condition relative to the condition corresponding to the at least one STANDARD assignee by the doctor;

a unit for assessing the patient's condition by the patient's ECG series, which assesses variation in the observed patient's condition over time by temporal variation in the distances between each newly received DECGs in the patient's DECG series and the assigned at least one STANDARD;

a unit for predicting the observed patient's condition, which predicts the most probable future condition of the patient on the basis of “history of observations” comprising recorded variations of past observed patient's conditions over time relative to the assigned at least one STANDARD;

a unit for imaging the patient's condition, which displays the dynamics of variation in distances of the patient's newly received ECGs as a displacement of cardiogram geometric paths between the assigned STANDARDS.

Preferably, the unit for predicting the observed patient's condition outputs a prognosis of the observed patient's condition in future periods of time, starting from the last instant of recording the patient's ECG, based on the type of variation in the observed patient's ECG path relative to the center of gravity of the assigned at least one STANDARD.

Preferably, the patient identifiers contain two sets of features, a first set of features defining the patient's personal data code, and a second set of features comprising parameters of the outpatient's card selected from the group consisting of features of gender, age, diagnosis, social status, blood type, profession, belonging to social groups, education, genotype, with/without pathology, taking/not taking medications.

Preferably, the form similarity measure between any ECG pairs is specified as the distance between their corresponding pairs of DECG points, calculated by formulas for calculating the distance between points in at least one selected space: Euclidean space, Riemannian space, Lobachevsky space, Hilbert space.

Preferably, the clustering method is selected from the group consisting of: K-means, K-medians, C-means, EM, FOREL, Kohonen neural network, graph methods, including single link methods, complete link methods, Ward method, average link methods, and weighted average link methods.

Preferably, the clustering unit checks, for the next analyzed ECG related to the extracted subset, the measure of similarity of its form with forms of all ECGs in each of the already formed clusters, and

if the analyzed ECG according to the predetermined form similarity measure matches at least one of the ECGs of one already formed cluster, then the ECG corresponds to this cluster and falls into this cluster;

if the ECG corresponds to more than one cluster, the previously formed clusters, to which the analyzed ECG corresponds, are merged into a single cluster;

if the ECG does not correspond to any of the previously formed clusters, a new cluster containing this single ECG is formed.

Preferably, in long-term ECGs a finite time interval from 1 to 5 minutes is selected, and DECG is determined in this time interval for further calculation of the similarity measure.

Preferably, in long-term ECGs more than one finite interval is selected; DECG is determined in these time intervals for further calculation of the similarity measure, and a path of patient's condition variation during the ECG recording procedure is constructed based on the similarity measure.

Preferably, the space of features is normalized by reducing the recorded ECGs to one fixed length using an interpolation method.

According to a second aspect of the invention the object of the invention is attained by providing a computer-assisted method for remote monitoring, analysis and prognosis of the patient's condition using a medical system according to claim 1, comprising:

gathering, for each patient from a plurality of observed patients, a series of patient's at least first-lead electrocardiograms (ECGs) marked with patient's identifiers in a database of said medical system, generated by adding each newly recorded patient's ECG marked with said identifiers to the patient's ECG series gathered previously throughout the entire observation period;

extracting, on doctor's request, from the gathered database an ECG subset of a target group of patients, which are combined into a target group in accordance with restrictions specified in the doctor's request and at least one of said identifiers in accordance with the examination purpose;

clustering the extracted ECG subset for the at least one target group of patients combined by at least one feature, for which purpose all the ECGs extracted to the subset are sequentially combined into at least one cluster by the feature of similarity between their forms using one of clustering methods;

determining a similarity measure of ECG forms in the space of ECG form features, for which purpose each ECG from the medical system database is associated with a discretized ECG (DECG), which is a point having coordinates in at least one space of ECG form features chosen by the system operator, and coordinates of the point in said space of ECG form features carry information about the original ECG form, and the measure of ECG form similarity between any ECG pairs is specified as the distance between their corresponding pairs of DECG points in this space;

generating a respective STANDARD for each cluster by forming a set of DECG points corresponding to the cluster ECGs and calculating characteristics of the formed DECG set, including: coordinates of the center of gravity of the formed DECG set, radius of the sphere or center of the polygon at the center of gravity of the DECG set, including all DECGs sets;

assigning at least one STANDARD from a set of generated standards for subsequent assessment of the patient's condition in accordance with the examination purpose specified by the doctor and comparing the patient's condition with the condition corresponding to the assigned at least one STANDARD;

assessing variation of the observed patient's condition over time by temporal variation in distances between each of the newly received DECGs in the patient's DECG series and the assigned at least one STANDARD;

predicting the observed patient's condition based on “observation history” comprising recorded variations in past observed patient's conditions over time relative to the assigned at least one STANDARD;

imaging the patient's condition as the dynamics of variation in the distances of the newly received patient ECGs at a displacement of geometric paths between the assigned STANDARDS.

Preferably, the observed patient's condition is predicted for future time periods, starting from the last instant of recording the patient's ECG, based on the type of variation in the observed patient's ECG path relative to the center of gravity of the assigned at least one STANDARD.

Preferably, the patient identifiers are selected from two sets of features, a first set of features defining the patient's personal data code, and a second set of features comprising parameters of the outpatient's card selected from the set consisting of features of gender, age, diagnosis, social status, blood type, profession, belonging to social groups, education, genotype, with/without pathology, taking/not taking medications.

Preferably, said recording of the ECG series is carried out according to a predetermined examination method, which provides for recording ECGs at specified instants and/or upon the occurrence of predetermined events associated with the patient's health status, patient's daily routine, taking medications or medical procedures.

Preferably, the form similarity measure between any ECG pairs is specified as the distance between their corresponding pairs of DECG points, calculated by formulas for calculating the distance between points in at least one space: Euclidean space, Riemannian space, Lobachevsky space, Hilbert space.

Preferably, the clustering method selected from the group consisting of: K-means, K-medians, C-means, EM, FOREL, Kohonen neural network, graph methods, including single link methods, complete link methods, Ward method, average link methods, and weighted average link methods.

Preferably, the next analyzed ECG related to the extracted subset is clustered, for which purpose the similarity measure of its form with forms of all ECGs in each of the already formed clusters is checked, and

if the analyzed ECG matches, by the predetermined form similarity measure, at least one of ECGs of the already formed one cluster, the ECG is considered as corresponding to this cluster and falling into this cluster;

if the ECG corresponds to more than one cluster, the previously formed clusters, to which the analyzed ECG corresponds, are merged into a single cluster;

if the ECG does not correspond to any of the previously formed clusters, a new cluster containing this single ECG is formed.

Preferably, the “with/without pathology” feature includes the presence or absence of pathology in the patient's diagnosis according to the International Classification of Diagnoses (ICD), confirmed by the doctor.

Preferably, the method further comprises using electrocardiograms selected from the set consisting of Eindhoven electrocardiogram, pulse wave cardiogram (photoplethysmogram), oxyhemogram, respiration card, echocardiogram, seismocardiogram and cardiovascular system signals synchronized with ECG.

### BRIEF DESCRIPTION OF THE DRAWINGS

The invention is further explained in the description of the preferred embodiment with reference to the accompanying drawings, in which:

FIG. 1 shows a schematic diagram of a medical system for remote monitoring, analysis and prognosis of the patient's condition, according to the invention;

FIG. 2 shows a scheme of forming STANDARDS;

FIG. 3 shows a diagram of a geometric path of newly arrived patient's ECGs between assigned STANDARDS;

FIG. 4 is a schematic diagram of gathering patient's ECGs by transmitting data from a cell phone, with data transmission over the mobile operator Internet.

### THEORETICAL BASIS OF THE PRESENT METHOD

In 1954, Fermi, Pasta, and Ulam made an attempt to demonstrate numerically that a nonlinear system inevitably transits from a state of single-mode excitation to a state with equally distributed energy.

The inventors proposed and mathematically described a variant of the Fermi-Pasta-Ulam recurrence model (FPU auto recurrence) and hypothesized an adequate description of the heart's electrical dynamics within the detected FPU auto recurrence phenomenon. The dynamics of the FPU auto recurrence making appropriate electrical dynamics of the normal functioning of the heart in the form of an electrocardiogram (ECG) was obtained by a computer model study (see A. V. Shmid, A. A. Berezin, M. A. Novopashin, Fermi-Pasta-Ulama auto recurrence in the description of the electrical activity of the heart, Journal Medical Hypothesis, 101 (2017) pp. 17-22).

The model studies have revealed a hypothetical basis of coronary heart disease in the form of increasing the energy of high-frequency harmonics spectrum of the FPU auto recurrence by reducing the energy of low-frequency harmonic spectrum of the auto recurrence, which ultimately leads to a sharp decrease in myocardial contractility.

The hypothesis was tested on more than 20,000 ECGs of both healthy individuals and patients with cardiovascular disease that have been studied. The studies revealed that the dynamics of electrical activity of a normally functioning heart can be interpreted by the FPU auto recurrence phenomenon, and coronary heart disease is a violation of the energy ratio between the low and high frequency harmonics of the FPU auto recurrence.

To simulate the ECG, it was taken into account that the heart functions in a self-oscillating mode, which implies the presence of a similar principle of dynamics in the FPU recurrence structure, used for simulating cardiac activity.

The mathematical simulation shows that electrical activity of the heart is the FPU auto recurrence, first formulated by the inventors, which has an individual nature and contains in its structure a picture of the physiological state of the heart and pathological processes whose patterns periodically appear in the Fourier spectra of auto recurrences.

Auto recurrences of Fourier spectra of healthy individuals ECGs comprise both a low-frequency part in the range of 1-5 Hz, corresponding to the canonical FPU recurrence, and a high-frequency part in the range of 20-35 Hz, corresponding to the high-frequency FPU recurrence. Therefore, ECG is a biological example of a full FPU auto recurrence.

Based on the simulation results, it was suggested that the set of frequency patterns obtained from different cardiograms of one patient or several patients does not have a chaotic distribution, and therefore several sets of frequency patterns can be distinguished by some measure of proximity.

This means that clustering algorithms can be applied to the normalized space of ECG frequency patterns, and stable frequency patterns of conditions of both a particular patient and a group of patients selected for a common feature can be obtained.

### Description of Preferred Embodiment

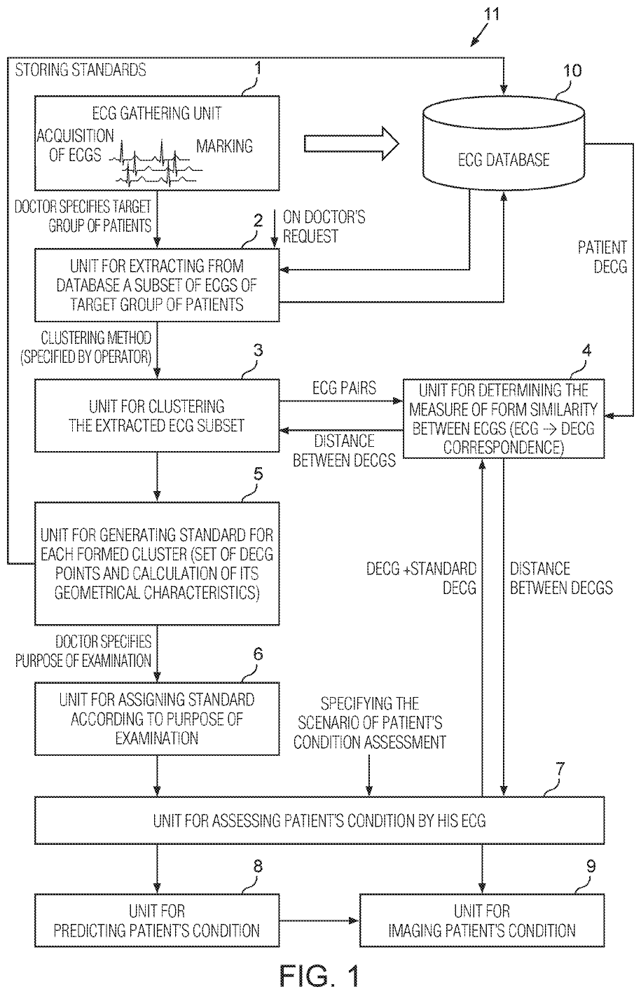

According to the invention, a medical system for remote monitoring, analysis and prognosis of the patient's condition by series of at least first-lead electrocardiograms (ECG) recorded for each patient from a plurality of observed patients comprises a unit 1 (FIG. 1) for gathering a series of patient's electrocardiograms (ECGs) in a database of said medical system for each of the plurality of observed patients. The electrocardiograms are marked with patient identifiers. Patient identifiers are selected from the set consisting of features of gender, age, diagnosis, social status, blood type, profession, belonging to social groups, education, genotype, with/without pathology, taking/not taking medications.

The series of electrocardiograms (ECGs) is generated by adding each new recorded patient's ECG marked with identifiers to the patient's ECG series gathered previously throughout the entire observation time.

The medical system further comprises a unit 2 for extracting, on doctor's request, from the gathered database a subset of ECGs belonging to a target group of patients, which are combined in the system into the target group in accordance with restrictions specified in the doctor's request and at least one of the identifiers in accordance with the examination purpose.

Each cardiogram received from the database is provided with a patient code and, if necessary, a medical identifier, i.e. it specifies who is being studied and what is being studied, for example, cardiograms of young individuals under the age of 30 who are constantly taking analgesics are being studied.

The medical system comprises a unit 3 for clustering the extracted subset of ECGs. The unit 3 sequentially combines, for at least one target group of patients combined by at least one identifier, all the ECGs extracted to the subset to at least one cluster A1, A2, A3, A4, A5 based on the similarity between their forms using a clustering method.

A conventional clustering method is used, which is selected from the set consisting of: K-means, K-medians, C-means, EM, FOREL, Kohonen neural network, graph methods, including single link methods, complete link methods, Ward method, average link methods, and weighted average link methods.

A unit 4 for determining a similarity measure of ECG forms determines the ECG form similarity measure in the space of ECG form features. From the clustering unit 3, the electrocardiograms are sequentially transferred in pairs to the unit 4 for determining the ECG form similarity measure to get a response i.e. the distance (similarity measure) between DECGs of the transferred ECG pair.

The unit 4 associates each ECG received from the medical system database and transferred from the clustering unit 3 with a discretized ECG (DECG), which is a point having coordinates in at least one space of ECG form features chosen by the system operator. Values of coordinates of the point in this ECG form feature space carry information about the form of the original ECG, and the form similarity measure between any ECG pairs is specified as the distance between their corresponding pairs of DECG points in this space. The space of similarity features is specified by the operator.

The clustering unit 3 checks, for the next analyzed ECG related to the extracted subset, the measure of its form similarity with forms of all ECGs in each of the already formed clusters A1 to A5.

If according to the predetermined form similarity measure the analyzed ECG matches at least one of the ECGs of one already formed cluster, then the ECG corresponds to this cluster and falls into this cluster. If the ECG corresponds to more than one cluster, then the previously formed clusters, which correspond to the analyzed ECG, are merged into a single cluster, for example, A3 is merged with A4. If the ECG does not correspond to any of the previously formed clusters, a new cluster A5 containing this single ECG is formed.

Furthermore, the measure of form similarity between any EGG pairs is specified as the distance between their corresponding pairs of DECG points, calculated by formulas for calculating the distance between points in at least one selected space: Euclidean space, Riemann space, Lobachevsky space, Hilbert space.

In long-term ECGs, a finite interval of 1 to 5 minutes is chosen, and DECG is determined in this interval for further calculation of the similarity measure.

In long-term ECGs, more than one finite interval is chosen, and the DECG is determined in these intervals for further calculation of the similarity measure, and the path of patient's condition variation during the procedure of recording the long-term ECGs is constructed on the basis of the similarity measure.

The feature space is normalized by reducing the recorded ECGs to one fixed length using interpolation method.

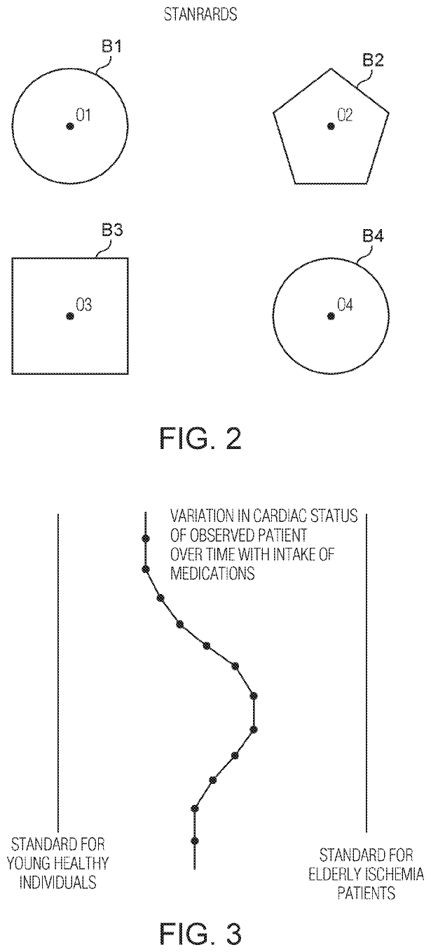

The medical system comprises a unit 5 for generating a respective STANDARD for each cluster A1 to A4 formed by the clustering unit 3 by forming a set of DECG points corresponding to the cluster ECGs, and calculating characteristics of the formed set of DECGs. The characteristics include: coordinates of the center of gravity 01, 02, 03, 04 of the generated DECG set, radius of the sphere or center of the polygon at the center of gravity of the DECG set, which includes all DECG sets. FIG. 2 shows STANDARDS B1, B2, B3 and B4.

The generated STANDARDS can be stores in the ECG database.

An assignment unit 6 assigns STANDARD on the doctor's request of at least STANDARD in accordance with the examination purpose for subsequent assessment of the patient's condition relative to the condition corresponding to the at least one STANDARD assigned by the doctor from the set of formed STANDARDS. Therefore, the assigned STANDARD corresponds to the condition indicated in the examination purpose. Optionally, the stored STANDARD can be received from the ECG database.

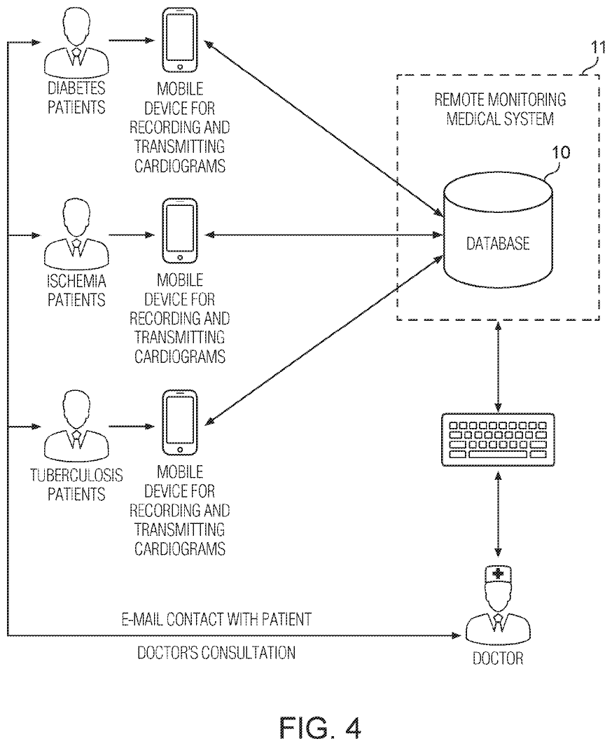

Then, a unit 7 for assessing the patient's condition by his ECG series assesses the variation in the observed patient's condition over time by temporal variation in the distance between each of the newly received DECGs in the patient's DECG series and the assigned at least one STANDARD. Since the STANDARD is formed, the doctor or the patient himself can compare the ECG series of one patient's, received from the database, with the STANDARD. FIG. 3 shows a DECG displacement diagram of the observed patient who is taking medications, where the left side shows the STANDARD of young healthy persons, the right side shows the STANDARD of elderly ischemia patients.

The present medical system provides prognosis by a unit 8 for predicting the observed patient's condition, in which the most probable future condition of the patient is predicted based on “observation history” comprising recorded variations of past conditions of the observed patient over time relative to the assigned at least one STANDARD.

The unit 8 for predicting the observed patient's condition outputs a prognosis of the observed patient's condition for future periods of time, starting from the last instant of recording the patient's ECG, based on the type of variation in the ECG path of the observed patient (FIG. 3) relative to the center of gravity of the assigned at least one STANDARD.

An imaging unit 9 displays the patient's condition, for which purpose the dynamics of variation in the distances of the newly received patient ECG is displayed as a displacement of geometric paths between the assigned STANDARDS (FIG. 3).

The medical system can also be applied to a group of patients.

The invention also provides a computer-assisted method for remote monitoring, analysis and prognosis of the patient's condition, which uses the medical system and compares forms of a set of patient's ECGs as carriers of information about the actual patient's health status.

In the present method, the computer first trains, i.e. generates a set of diagnosis STANDARDS, each comprising a multitude of similar in form ECGs of previously observed patients, corresponding to one of the STANDARDS set by the doctor, characterizing the alleged diagnosis.

Further, the trained computer is used to assess the measure of ECG form proximity of a patient with an unknown diagnosis with one of the STANDARDS formed in the course of computer training.

Current condition or the patient is assessed by the measure of proximity of his ECG form to the ECG forms of one or more STANDARDS.

Variation in the patient's condition over time is assessed based on the analysis of his ECG series whether the patient's ECG form similarity measure is approaching or separating from the ECG STANDARD forms.

Prognosis of the patient's condition progression is made on the basis of analysis of the history of variation in the form similarity measure of his ECG series relative to the ECG STANDARD forms.

Measurement of the ECG form similarity measure relies on the ability to represent each of the patient's ECGs as a point (DECG) with coordinates that reflect the ECG form in a selected space of ECG form similarity features, The introduction of the space of ECG form similarity features makes it possible to further operate with geometric terms, in other words, to quantify the ECG form similarity measure as the distance between points (DECG), and consider the diagnosis STANDARD as a set of points, i.e. an area in the space of form similarity features. Therefore, the method enables the transition from a visual informal assessment of the ECG form as the basis of diagnosis to a formal computer-assisted assessment.

Hereinafter are steps of a method for remote monitoring, analysis and prognosis of the patient's condition using first-lead electrocardiogram series (ECG) recorded for one of the observed patients.

To implement the method, it is initially required to gather, for each patient from a plurality of observed patients in the medical system database, a series of at least first-lead electrocardiograms (ECGs) marked with patient identifiers. The series of electrocardiograms (ECGs) is generated by adding each newly recorded ECG of a patient from the observed plurality of patients, marked with identifiers, to the patient's ECG series gathered previously throughout the entire observation time. Thus, the database comprises a multitude of at least first-lead electrocardiograms (ECGs).

The electrocardiograms are marked with patient identifiers, Patient identifiers are selected from the features of sex, age, diagnosis, social status, blood type, profession, belonging to social groups, education, genotype, with/without pathology, taking/not taking medications.

Then, in accordance with doctor's request, the unit 3 extracts a subset of ECGs of the target group of patients from the gathered database. Patients are combined into the target group in accordance with restrictions specified in the doctor's request and at least one of the identifiers pursuant to the examination purpose. For example, cardiograms of young individuals under the age of 30 years who are constantly taking analgesics are studied. Or a doctor may request all the patients taking a set of medications.

Each cardiogram received from the database is provided with a patient code and, if necessary, a medical identifier, i.e. contains information about who is being studied and what is being studied.

Through this process a subset of ECGs of the target patient group is generated.

Then, the unit 3 performs clustering of the extracted ECG subset of the target group of patients combined by at least one identifier, for this purpose all the ECGs extracted to the subset are sequentially combined into at least one cluster on the basis of similarity between their forms using one of clustering methods.

Clustering method is selected from the set consisting of K-means, K-medians, C-means, EM, FOREL, Kohonen neural network, graph methods, including single link methods, complete link methods, Ward method, average link methods, weighted average link method.

The unit 4 for determining ECG form similarity measure determines ECG form similarity measures in the space of ECG form features. Electrocardiograms from the clustering unit are transferred in pairs to the ECG form similarity measure determining unit 4 to receive a response, i.e. the distance (similarity measure) between DECGs of the transferred ECG pair.

The unit 4 associates each ECG received from the medical system database and transferred from the clustering unit 3 a discretized ECG (DECG), which is a point having coordinates in at least one ECG form feature space chosen by the system operator. Values of coordinates of the points in the ECG form feature space carry information about the form of the original ECG, and the measure of similarity between forms of any ECG pairs is specified as the distance between their corresponding pairs of DECG points in this space. The similarity features space is specified by the operator.

As stated above, all the ECGs extracted to a subset are sequentially combined into at least one cluster based on the similarity between their forms.

The clustering unit 3 checks, for the next analyzed ECG related to the extracted subset, the measure of similarity of its form with the forms of all ECGs in each of the already formed clusters A1 to A5.

If the analyzed ECG matches, by the predetermined form similarity measure, least one of the ECGs of the already formed one cluster, the ECG is considered as corresponding to this cluster and, therefore, falling into this cluster. If the ECG corresponds to more than one cluster, the previously formed clusters, to which the analyzed EGG corresponds, are merged into a single cluster, e.g. A3 is merged with A4. If the ECG does not correspond to any of the previously formed clusters, a new cluster A5 containing this single ECG is formed (FIG. 2).

Furthermore, the measure of similarity of forms between any ECG pairs is specified as the distance between their corresponding pairs of DECG points, calculated by formulas for calculating the distance between points in at least one selected space: Euclidean space, Riemann space, Lobachevsky space, Hilbert space.

In long-term ECGs, a finite interval of 1 to 5 minutes is chosen, and DECG is determined in this interval for further calculation of the similarity measure.

In long-term ECGs, more than one finite interval is chosen, and DECG is determined in these intervals for further calculation of the similarity measure, and, the path of the patient's condition variation is constructed based on the similarity measure during the procedure of recording the long-term ECGs.

The space of features is normalized by reducing the recorded ECGs to one fixed length using an interpolation method.

Then, the STANDARD generation unit 5 generates a respective STANDARD for each cluster. The unit 5 generates a respective STANDARD B1 to B4 for each cluster A1 to A4 formed by the clustering unit 3 by forming a set of DECG points corresponding to ECGs of the cluster, and calculates characteristics of the generated set of DECG. The characteristics include: coordinates of the center of gravity 01, 02, 03, 04 (FIG. 2) of the generated DECG set, radius of the sphere or center of the polygon at the center of gravity of the DECG set, which includes all DECGs of the set.

The generated STANDARDS can be stored in the ECG database.

For subsequent assessment of the patient's condition, at least one STANDARD is assigned in accordance with the examination purpose. STANDARD is assigned by the STANDARD as unit 6 on doctor's request in accordance with the examination purpose for subsequent assessment of the patient's condition relative to the condition corresponding to the doctor-assigned at least one STANDARD from the set of formed STANDARDs.

Variation in the observed patient's condition over time is evaluated by temporal variations in distances between each of the newly received DECGs in the patient's DECG series and the assigned at least one STANDARD.

Therefore, the assigned STANDARD corresponds to the condition indicated in the objective of examination.

Optionally, the stored STANDARD can be received from the ECG database.

Then, the observed patient ECG series is fed from the ECG database to the patient's condition assessment unit 7, where the variation in the observed patient's condition over time is evaluated by temporal variation in the distance between each of the newly received DECGs in the patient's DECG series and the assigned at least one STANDARD. Since the STANDARD is formed, the doctor or patient can compare one patient's ECG series received from the database with the at least one STANDARD.

The doctor's request is entered electronically by conventional methods.

It is important for the treating doctor and the patient to know the prognosis, for example, how the medication dosage must be adjusted depending on the observed patient's condition and how the patient's condition will change in future if the medication dosage is maintained, or whether a change in the dosage is required.

The present medical system provides the prognosis by the observed patient s condition prognosis unit 8, which predicts the most probable future condition of the patient on the basis of “observation history” comprising recorded temporal variations in past conditions of the observed patient relative to the assigned at least one STANDARD.

The observed patient's condition is predicted for future periods of time, starting from the last instant of recording the patient's ECG, based on the type of variation in the observed patient ECG path relative to the center of gravity of the assigned at least one STANDARD.

The medical system provides imaging of the patient's condition as the dynamics of variation in the distance of the patient's newly received ECGs at a displacement of geometric paths between the assigned STANDARDS.

The ECG series is recorded according to a predetermined examination method providing for recording ECGs at predetermined instants and/or upon occurrence of predetermined events related to patient's health status, patient's daily routine, medication intake or medical procedures.

The “with/without pathology” feature includes the presence or absence of pathology in the patient's diagnosis according to the International Classification of Diagnoses (ICD), confirmed by the doctor.

Optionally, electrocardiograms from the set consisting of Einthoven electrocardiogram, pulse wave cardiogram (photoplethysmogram), oxyhemogram, respiration card, echocardiogram, seismocardiogram and cardiovascular system signals, synchronized with ECGs can be used.

### EXAMPLE

FIG. 3 shows schematically a variation in the patient's condition as the dynamics of variation in the distances of the newly received patient ECGs at a displacement of geometric paths between assigned STANDARDS.

The left side of FIG. 3 shows, by a vertical line, a previously received STANDARD of cardiograms belonging to young healthy individuals under 30 years of age who do not take any medications, and the right side shows a line for a previously received STANDARD of cardiograms belonging elderly individuals over 60 years of age who take cardiac medications.

A patient takes his first-lead electrocardiogram several times a day (e.g. five times) using the cell phone application, and transfers data from the cell phone over the mobile operator's Internet to the database. From the database, the cardiograms are sequentially entered into the remote monitoring medical system at the request of the doctor who monitors and analyzes the patient's condition.

Upon receipt, the medical system processes each cardiogram, i.e. assigns it to a target group of patients, performs clustering, determines its similarity measure, forms a new STANDARD if necessary, and inputs in accordance with the STANDARD assigned by the doctor into the patient condition assessment unit, where the last received ECG is compared with the STANDARD. The comparison result is transferred to the unit for predicting the observed patient's condition by electrocardiograms (ECGs) series, which outputs a prognosis of the patient's future condition based on “observation history” comprising recorded variations in past conditions of the observed patient over time relative to the assigned at least one STANDARD.

The prognosis result enters the imaging unit, which displays the patient's condition as the dynamics of variations in the distance of the newly received patient's ECGs at a displacement of geometric paths between the assigned STANDARDS (FIG. 3).

### INDUSTRIAL APPLICABILITY

The present computer-assisted method for monitoring, analysis and prognosis of the patient's condition by series of at least first-lead electrocardiograms (ECGs) and other cardiologic signals synchronized with the ECG provides an effective diagnosis of patients outside the healthcare center when determining the patient's condition and renders aid to both the patient in express assessment of his current condition and to the clinician in monitoring the course of treatment of the patient outside the healthcare center, including the assessment of cardiotoxicity in the application of medications, not only cardiac ones.

1. A medical system for remote monitoring, analysis and prognosis of the patient's condition by series of at least first-lead electrocardiograms (ECGs) recorded for each patient from a plurality of observed patients, comprising:
a unit (1) for gathering in a database of said medical system for each of the observed patients a series of his electrocardiograms (ECGs) marked with patient identifiers, generated by adding each newly recorded patient's ECG marked with said identifiers to the patient's ECG series gathered previously throughout the entire observation time;
a unit (2) for extracting on doctor's request from the gathered database an ECG subset of a target group of patients, which are combined in said system into the target group in accordance with restrictions specified in the doctor's request and at least one of said identifiers in accordance with the examination purpose;
a unit (3) for clustering the extracted ECG subset, which sequentially combines, for at least one target group of patients combined by at least one feature, all the ECGs extracted to the subset to at least one cluster based on the feature of similarity of their forms using a clustering method;
a unit (4) for determining an ECG form similarity measure in the space of ECG form features, which associates each ECG from the database of the medical system with a discretized ECG (DECG), which is a point having coordinates in at least one space of ECG form features, chosen by the system operator, wherein coordinates of the point in said space of ECG form features carry information about the original ECG form, and the ECG form similarity measure between any ECG pairs is specified as the distance between their corresponding pairs of DECG points in this space;
a unit (5) for generating STANDARDS, which generates, for each cluster formed by the unit (3), a corresponding STANDARD by forming a set of DECG points corresponding to the cluster ECGs, and calculates characteristics of the formed DECG set, including: coordinates of the center of gravity of the formed DECG set, radius of the sphere or center of the polygon at the center of gravity of the DECG set, including all the DECG sets;
a unit (6) for assigning, on doctor's request, at least one STANDARD in accordance with the examination purpose from a plurality of generated STANDARDS for subsequent assessment of the patient's condition relative to the condition corresponding to the at least one STANDARD assigned by the doctor;
a unit (7) for assessing the patient's condition by the patient's ECG series, which assesses variation in the observed patient's condition over time by temporal variation in the distances between each newly received DECGs in the patient's DECG series and the assigned at least one STANDARD;
a unit (8) for predicting the observed patient's condition, which predicts the most probable future condition of the patient on the basis of “history of observations” comprising recorded variations of past observed patient's conditions over time relative to the assigned at least one STANDARD;
a unit (9) for imaging the patient's condition, which displays the dynamics of variation in distances of the patient's newly received ECGs as a displacement of cardiogram geometric paths between the assigned STANDARDS.

2. The medical system according to claim 1, wherein the unit (8) for predicting the observed patient's condition outputs a prognosis of the observed patient's condition in future periods of time, starting from the last instant of recording the patient's ECG, based on the type of variation in the observed patient's ECG path relative to the center of gravity of the assigned at least one STANDARD.

3. The medical system according to claim 1, wherein the patient identifiers contain two sets of features, a first set of features defining the patient's personal data code, and a second set of features comprising parameters of the outpatient's card selected from the group consisting of features of gender, age, diagnosis, social status, blood type, profession, belonging to social groups, education, genotype, with/without pathology, taking/not taking medications.

4. The medical system according to claim 1, wherein the form similarity measure between any ECG pairs is specified as the distance between their corresponding pairs of DECG points, calculated by formulas for calculating the distance between points in at least one selected space: Euclidean space, Riemannian space, Lobachevsky space, Hilbert space.

5. The medical system according to claim 1, wherein the clustering method is selected from the group consisting of: K-means, K-medians, C-means, EM, FOREL, Kohonen neural network, graph methods, including single link methods, complete link methods, Ward method, average link methods, weighted average link methods.

6. The medical system according to claim 1, wherein said clustering unit (3) checks, for the next analyzed ECG related to the extracted subset, the measure of similarity of its form with forms of all ECGs each of the already formed clusters, and
if the analyzed ECG according to the predetermined form similarity measure matches at least one of the ECGs of one already formed cluster, then the ECG. corresponds to this cluster and falls into this cluster;
if the ECG corresponds to more than one cluster, the previously formed clusters, to which the analyzed ECG corresponds, are merged into a single cluster;
if the EGG does not correspond to any of the previously formed clusters, a new cluster containing this single ECG is formed.

7. The medical system according to claim 1, wherein in long-term ECGs a finite time interval from 1 to 5 minutes is selected, and DECG is determined in this time interval for further calculation of the similarity measure.

8. The medical system according to claim 1, wherein in long-term ECGs more than one finite interval is selected; DECG is determined in these time intervals for further calculation of the similarity measure, and a path of patient's condition variation during the ECG recording procedure is constructed based on the similarity measure.

9. The medical system according to claim 1, wherein the space of features is normalized by reducing the recorded ECGs to one fixed length using an interpolation method.

10. The medical system according to claim 1, wherein characteristics of the generated DECG set include: coordinates of the center of gravity of the generated DECG set, radius of the sphere or center of the polygon at the center of gravity of the DECG set, including ail the DECG sets.

11. A computer-assisted method for remote monitoring, analysis and prognosis of the patient's condition using a medical system according to claim 1, comprising:
gathering, for each patient from a plurality of observed patients, a series of patient's at least first-lead electrocardiograms (ECGs) marked with patient's identifiers in a database of said medical system, generated by adding each newly recorded patient's ECG marked with said identifiers to the patient's ECG series gathered previously throughout the entire observation period;
extracting, on doctor's request, from the gathered database an ECG subset of a target group of patients, which are combined into a target group in accordance with restrictions specified in the doctor's request and at least one of said identifiers in accordance with the examination purpose;
clustering the extracted ECG subset for the at least one target group of patients combined by at least one feature, for which purpose all the ECGs extracted to the subset are sequentially combined into at least one cluster by the feature of similarity between their forms using one of clustering methods;
determining a similarity measure of ECG forms in the space of ECG form features, for which purpose each ECG from the medical system database is associated with a discretized ECG (DECG), which is a point having coordinates in at least one space of ECG form features chosen by the system operator, and coordinates of the point in said space of ECG form features carry information about the original ECG form, and the measure of ECG form similarity between any ECG pairs is specified as the distance between their corresponding pairs of DECG points in this space;
generating a respective STANDARD for each cluster by forming a set of DECG points corresponding to the cluster ECGs and calculating characteristics of the formed DECG set, including: coordinates of the center of gravity of the formed DECG set, radius of the sphere or center of the polygon at the center of gravity of the DECG set, including all DECGs sets;
assigning at least one STANDARD from a set of generated standards for subsequent assessment of the patient's condition in accordance with the examination purpose specified by the doctor and comparing the patient's condition with the condition corresponding to the assigned at least one STANDARD;
assessing variation of the observed patient's condition over time by temporal variation in distances between each of the newly received DECGs in the patient's DECG series and the assigned at least one STANDARD;
predicting the observed patient's condition based on “observation history” comprising recorded variations in the past observed patient's conditions over time relative to the assigned at least one STANDARD;
imaging the patient's condition as the dynamics of variation in the distances of the newly received patient ECGs at a displacement of geometric paths between the assigned STANDARDS.

12. The method according to claim 11, wherein the observed patient's condition is predicted for future time periods, starting from the last instant of recording the patient's ECG, based on the type of variation in the observed patient's ECG path relative to the center of gravity of the assigned at least one STANDARD.

13. The method according to claim 11, wherein the patient identifiers are selected from two sets of features, a first set of features defining the patient's personal data code, and a second set of features comprising parameters of the outpatient's card selected from the set consisting of features of gender, age, diagnosis, social status, blood type, profession, belonging to social groups, education, genotype, with/without pathology, taking/not taking medications.

14. The method according to claim 11, wherein said recording of the ECG series is carried out according to a predetermined examination method, which provides for recording ECGs at specified instants and/or upon the occurrence of predetermined events associated with the patient's health status, patient's daily routine, taking medications or medical procedures.

15. The method according to claim 11, wherein the form similarity measure between any ECG pairs is specified as the distance between their corresponding pairs of DECG points, calculated by formulas for calculating the distance between points in at least one space: Euclidean space, Riemannian space, Lobachevsky space, Hilbert space.

16. The method according to claim 11, wherein the clustering method is selected from the group consisting of: K-means, K-medians, C-means, EM, FOREL, Kohonen neural network, graph methods, including single link methods, complete link methods, Ward method, average link methods, weighted average link methods.

17. The method according to claim 11, wherein the next analyzed ECG related to the extracted subset is clustered, for which purpose the similarity measure of its form with forms of all ECGs in each of the already formed clusters is checked, and
if the analyzed ECG matches, by the predetermined form similarity measure, at least one of ECGs of the already formed one cluster, the ECG is considered as corresponding to this cluster and falling into this cluster;
if the ECG corresponds to more than one cluster, the previously formed clusters, to which the analyzed ECG corresponds, are merged into a single cluster;
if the ECG does not correspond to any of the previously formed clusters, a new cluster containing this single ECG is formed.

18. The method according to claim 11, wherein the “with/without pathology” feature includes the presence or absence of pathology in the patient's diagnosis according to the International Classification of Diagnoses (ICD), confirmed by the doctor.

19. The method according to claim 11, further comprising using electrocardiograms selected from the set consisting of Eindhoven electrocardiogram, pulse wave cardiogram (photoplethysmogram), oxyhemogram, respiration card, echocardiogram, seismocardiogram and cardiovascular system signals synchronized with ECG.

20. The method according to claim 11, wherein characteristics of the generated DECG set include: coordinates of the center of gravity of the generated DECG set, radius of the sphere or center of the polygon at the center of gravity of the DECG set, including all DECG sets.
